(19) United States
(12) Patent Application Publication
(10) Pub. No.: US 2012/0310664 A1
(43) Pub. Date: Dec. 6, 2012

(54) System and Method for Monitoring Hospital Workflow Compliance with a Hand Hygiene Network

(76) Inventors: Avery Dallas Long, Madison, AL (US); Harvey Allen Nix, Birmingham, AL (US); Feihong Xin, Hoover, AL (US); Romeo Maurice Burtis, Birmingham, AL (US)

(21) Appl. No.: 13/149,283

(22) Filed: May 31, 2011

(51) Int. Cl. G06Q 50/22; G06Q 50/24

(52) U.S. Cl. 705/3; 705/2

(57) ABSTRACT

A system and method for monitoring compliance with a plurality of workflow procedures in a hospital or other health care facility using a hand hygiene compliance system (HHC). A control unit of an HHC gathers data based upon the presence, identification, and movement of a plurality of assets, including persons, equipment, or supplies, having wearable detectable tags, such as RFID tages, and communicates that data to a local or remote server. The server is programmed to monitor or cause compliance with hospital workflow procedures relevant to the communicated data, such as rounding requirements and proper use of equipment, or may generate alarms or notifications where a workflow procedure has not been followed.

### FIELD OF THE INVENTION

The present invention relates to the use of a communications network created by a hand hygiene compliance (HHC) system in conjunction with a plurality of wearable tags on hospital personnel and other assets to monitor or cause compliance with hospital workflow procedures.

### BACKGROUND

The issue of hospital acquired infections is well known within and outside the health care community. Such infections kill more Americans each year than AIDS, breast cancer, and automobile accidents combined. To date many studies have been conducted in an effort to ascertain effective ways to reduce the occurrence of such infections, and the clear majority finds a thorough cleansing of one's hands prior to treating a patient as the single most important way to protect against the spread of hospital-acquired infections. As a result, many hospitals have implemented HHC systems for purposes of monitoring whether such persons wash their hands upon entering a patient's room. As such, HHC systems monitoring hand hygiene compliance are well established in the prior art.

However, the communication networks created by the aforementioned HHC systems generally monitor and report only hygiene-related events. As a result, these communication networks do not monitor non-hygiene events associated with a piece of equipment (i.e., a catheter), a supply, or a person for purposes of providing information relevant to hospital procedures, such as workflow procedures, based on data related to the piece of equipment, the supply, or the person. If HHC systems were improved to provide such non-hygiene related information, patient care would increase due to an increase in compliance with hospital workflow procedures which would result in an even greater overall reduction in the number of hospital-acquired infections. While current HHC systems are effective in monitoring hand hygiene compliance, they do not monitor and provide information related to various non-hygiene events (i.e. hospital workflow procedures).

### SUMMARY

Embodiments of the present invention provide a system for monitoring compliance with a plurality of workflow procedures in a hospital or other health care facility using an HHC system. The system includes a HHC system, which provides a communications network capable of detecting the presence of a hospital employee having a wearable tag, preferably in the form of a Radio Frequency Identification (RFID) tag, and monitoring whether the hospital employee washed his hands upon entering a patient's room. Each HHC control unit (that is, a hand washing station equipped with a sensor and communication devices) is provided with a feedback device in the form of a small display and necessary hardware to communicate with the wearable tag and a communications network, such as a wireless computer network. Through the communications network, the control unit is in communication with computing devices throughout the hospital, including, for example, servers or personal computers at an administrator's desk or nurses' stations. The display associated with each server or computer associated with the communication network operates as a feedback device in embodiments of the present invention.

In one embodiment, the control units gather data based upon the presence, identification, and movement of a plurality of assets having the wearable tags, and communicating that data to a local or remote server. The assets monitored by the HHC system may be persons, equipment, or supplies. The server is programmed to monitor or cause compliance with hospital workflow procedures. For example, the system may generate a report of compliance with hospital procedure from data gathered by the HHC system in response to a query by a user and display the report of compliance on a feedback device located in the hospital. One such report may include a hospital rounding report based upon the movement of hospital personnel (i.e. nurses, doctors, security guards) equipped with RFID tags, into rooms equipped with a HHC system. The system also may automatically select one or more hospital procedures relevant to the content of data gathered by the HHC and provide workflow instruction through a feedback device to a person performing such procedure to ensure compliance with the procedure. For example, a control unit may detect the presence of an RFID-tagged piece of equipment, such as a catheter, in a room, and in response to movement of the catheter by a RFID-tagged nurse, display a procedure on a feedback device associated with the control unit relating to proper administration of the catheter and further requiring input confirming compliance with the procedure by the nurse. The system also may automatically generate an alarm or notification signifying a procedure is not currently being followed, in response to data gathered by the HHC. For example, the system may dynamically update patient rounding status, including based upon rounding intervals specific to individual patents based upon their health status, and in a preferred embodiment display a red screen or audible warning on a feedback device at a nurses' workstation when the maximum time between visits to a patient's room has elapsed.

These and other embodiments of the present invention will become readily apparent to those skilled in the art from the following detailed description of the embodiments having reference to the attached figures, the invention not being limited to any particular embodiment(s) disclosed.

### BRIEF DESCRIPTION OF THE DRAWINGS

FIG. 1 depicts an exemplary plan view of a hospital floor equipped with an HHC system tracking movement of a nurse having a wearable tag performing patient rounding.

FIG. 2 is a flowchart illustrating one embodiment of a process of using data gathered by a HHC system to generate an automated rounding report.

FIG. 3 is an exemplary interface of an embodiment of the present invention for generating rounding reports for a subset of employees based upon data gathered by an HHC system.

FIG. 4 is an exemplary interface of an embodiment of the present invention for generating rounding reports for an individual employee within a subset of employees from data gathered by an HHC system.

FIG. 5 is an exemplary embodiment of a dynamic rounding report displayed on a feedback device located within a healthcare facility generated by an embodiment of the present invention from data gathered by an HHC system.

FIG. 6 is an exploded view of the dynamic rounding report depicted in FIG. 5 further illustrating a nurse or doctor's ability to view data relevant to patient rounding.

### DETAILED DESCRIPTION OF PREFERRED EMBODIMENTS

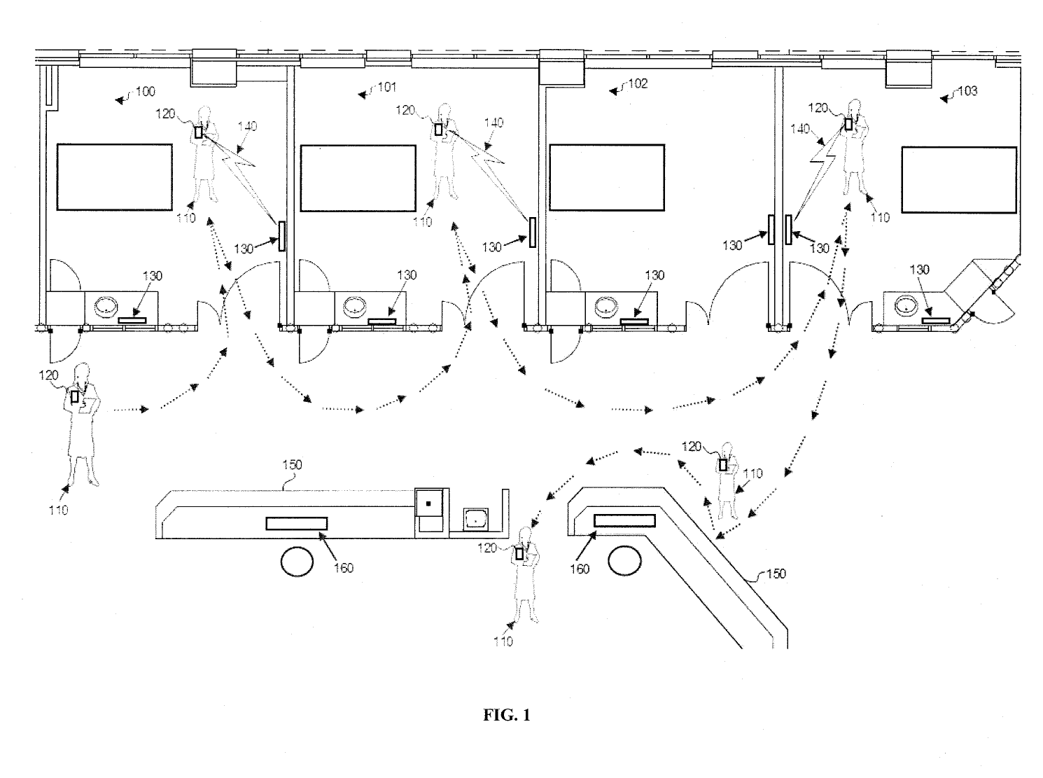

The various embodiments of the present invention and their advantages may be understood by referring to FIGS. 1 through 6 of the drawings. The elements of the drawings are not necessarily to scale, emphasis instead being placed upon illustrating the principles of preferred embodiments of the present invention. Throughout the drawings, like numerals are used for like and corresponding parts of the various drawings. This invention may be provided in other specific forms and embodiments without departing from the essential characteristics as described herein. The embodiments described below are to be considered in all aspects as illustrative only and not restrictive in any manner.

The present invention relates to a system comprising hand hygiene compliance (HHC) system comprising a plurality of control units, a plurality of wearable tags, and a server. The wearable tags, which in a preferred embodiment are in the form of a plurality of RFID tags, are associated with an asset consisting of a person, a piece of equipment, and a supply. The control units are operable to detect and identify wearable tags within a predetermined proximity of the control units and communicate data over a communications network associated with the HHC system to the server. Upon receiving the data, the server is operable at least in part on the data to perform at least one of the following actions: generate a report of compliance with a hospital procedure in response to a query by a user and display the report of compliance on a feedback device; select at least one hospital procedure relevant to the data and provide workflow instructions to a person performing such procedure; or generate a notification regarding compliance with a hospital procedure.

As used herein, the term “server” broadly refers to any computing device with a processor programmed to perform the functions described herein, and may include without limitation traditional servers, desktop or notebook computers, tablets, smart phones or PDAs, and any like device now existing or hereinafter developed. Likewise, the term “feedback” device broadly refers to any visual, auditory, or tactile device capable of conveying information to a person, including displays associated with HHC control units or displays of servers (as previously defined), or displays of workstation or client computers or devices receiving reports or other information from servers to convey to relevant healthcare providers. Further, the basic components and operation of an HHC system are known to those of ordinary skill in the art and will not be described in detail here.

Referring now to FIG. 1, a plan view of a hospital floor equipped with the hand hygiene compliance system is depicted having a plurality of rooms, shown generally as (100), (101), (102), and (103). A nurse (110) having a wearable tag (120) is shown performing patient rounding by entering and exiting room (100), room (101), and room (103). In this particular embodiment, a plurality of control units (130) detect movement of the nurse (110) in each of the aforementioned rooms by way of a wireless transmission (140) occurring between the wearable tag (120) and the control units (130). The wireless transmission (140) occurs when the wearable tag (120) associated with the nurse (110) enters a predetermined proximity of the control unit (130). In this illustrative embodiment, the wearable tag (120) enters the predetermined proximity upon the nurse (110) entering room (101), room (102), and room (103) and triggers the occurrence of the wireless transmission (140).

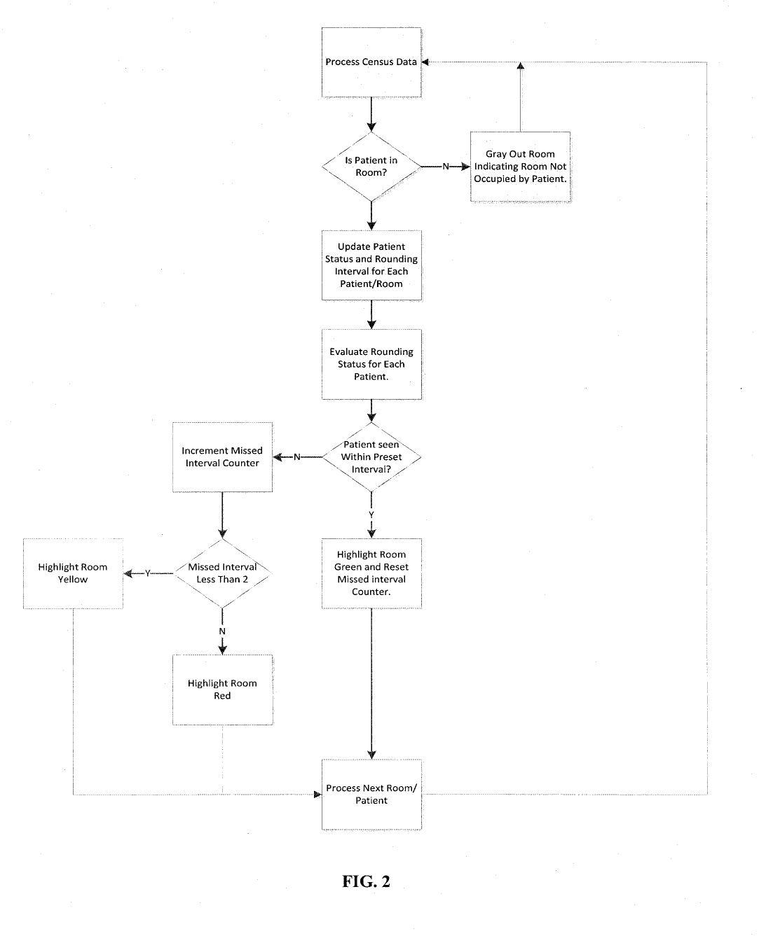

Still referring to FIG. 1, the control units (130) obtains via the wireless transmission (140) data relating to location, movement, time of detection, signal strength, and identity of the wearable tag (120) worn by the nurse (110) and subsequently transfers the data to a server, not shown in FIG. 1, through a communications network associated with the HHC system. As will be discussed in detail in subsequent FIG. 2, in one embodiment, the server in response to a query by a user is operable to generate a report of compliance with a hospital procedure based at least in part on data received from the wireless transmission (140) and further displays the report of compliance on a feedback device (160). For example, as shown generally in FIG. 1, a nurse (110), upon completing patient rounding, enters a nurses' work station (150) for purposes of consulting the feedback device (160) for a report of compliance with a hospital procedure relating to patient rounding. However, while this particular embodiment illustrates the feedback device (160) located at a nurses' work station (150), the feedback device (160) may be located at other locations of the hospital floor such as an administrator's desk, or remotely in connection with any server or computing device in communication with the hospital's information systems via a local or wide-area communications network, including the internet.

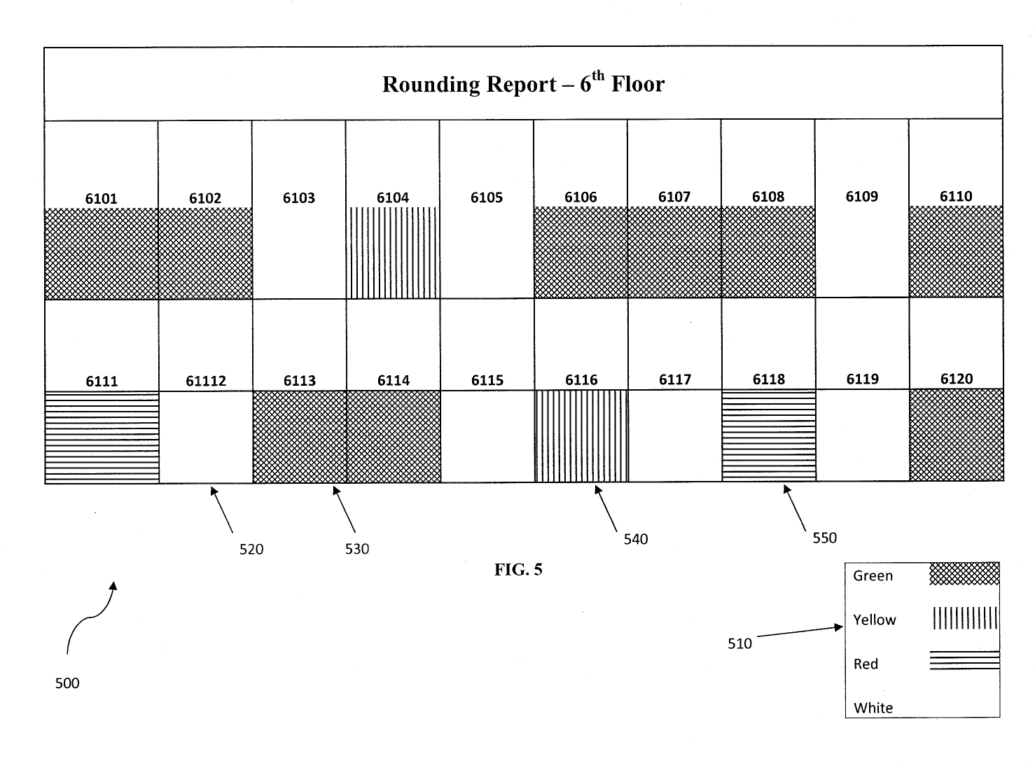

Turning to FIG. 2, a flowchart of one embodiment is provided depicting processes the server performs while generating a report of compliance with a hospital procedure. In this flowchart, the processes the server performs relate to generating an automated rounding report for a plurality of patients in a hospital, with the automated rounding report subsequently depicted in FIG. 5. When generating the automated rounding report, the server begins by accessing an HHS Census database, which allows the server to ascertain a timestamp indicating when a patient was admitted and a timestamp indicating when a patient was discharged. Furthermore, the HHS Census database also tells the server a location (i.e. a room number) of a patient assuming the patient has not been discharged. After acquiring these parameters, the server logically matches each of a plurality of rooms on a floor of a hospital with each of the patients contained in the HHS Census database. However, in the event the HHS Census database contains no record of a patient being admitted to a room on a floor in the hospital, the server designates the room as unoccupied, for example by displaying the room as a particular color (e.g., white) in the automated rounding report. The server regularly monitors the HHS Census database, so the automated rounding report generated remains dynamic and false alarms are not generated based upon a patient who has been discharged from her room.

Referring still to FIG. 2, the server next performs a status check for each of the patients by accessing a patient information database. The status check provides health condition data of each of the patients, for example whether the patients are in good, fair, or critical condition, or other indicia adopted by a particular hospital, department, or floor, as indicated by corresponding records in the patient information database. A rounding interval is assigned for each of the patients based upon the health condition data gathered from the records of the patient information database. The rounding interval may vary depending on this information. Generally, a hospital's procedures relating to patient rounding requires a member of hospital personnel to visit each of the patients on an hourly basis. However, a hospital's procedures may require more frequent visits by hospital personnel in the event heightened care is required. In addition, in a preferred embodiment, an authorized user may override the rounding interval assigned to provide a more specific rounding interval for a given patient.

Referring now to FIG. 2 in conjunction with FIG. 1, after a rounding interval for each of the patients contained in the HHS Census database has been assigned, the server accesses data received from the control units (130) located in each of the rooms occupied by each of the patients. Then the server analyzes the data to ascertain a rounding status for each of the patients. Using FIG. 1 as an example, the rounding status tells the server how long it has been since the nurse (110) having the wearable tag (120) entered each of the rooms occupied by each of the patients, based upon the time of detection of the wearable tag (120) by the control units (130). In FIG. 1, the rounding status for room (100), room (101), and room (103) would depict the nurse (110) as having been the last wearable tag (120) to visit. However, room (102) may have a rounding status separate and distinct from room (100), room (101), and room (103) since the nurse (110) did not enter room (102) while performing patient rounding.

Once the server determines a rounding status for each of the patients, the server compares the rounding status for each of the patients against the rounding interval for each of the patients and assigns a corresponding compliance indicator to each patient. The compliance indicator, as its name suggests, is a cue communicating the extent to which the patient's rounding status is in compliance with hospital procedure. The compliance indicator may be a numeric value (such as a range from 1 to 10), a “star” system (such as 1 to 5 stars), a color range (green, yellow, red), or any other similar system for conveying information. The compliance indicator also may utilize sound, such as a beep of varying frequency or interval based upon the rounding status of the reported patients. In a preferred embodiment, a color system is used, with a color block corresponding to each room, as such a format can be seen and understood from a distance, without having to read any text. Further, in a still preferred embodiment, a compliance factor for various levels of compliance or non-compliance may be assigned and adjusted. The compliance factor controls the state change of the compliance indicator with respect the extent of compliance with the hospital procedure. The compliance indicator may be binary (compliant or non-compliant), or it may have three or more states (complaint, varying degrees of non-compliance, urgent noncompliance). In the case of rounding, a particular department may have defined the compliance indicator as green (“compliant), yellow (“nearing non-compliant” or “moderately non-compliant”), and red (“urgent non-compliance”). The department may wish the compliance indicator to change from “compliant” (e.g., green) to “nearing non-compliant” (e.g., yellow) when the rounding interval is 90% complete (e.g., for one-hour rounding interval, 54 minutes into a since the last round by a nurse), and change from “nearing non-compliant” to “non-compliant” (e.g., “red”) when the 110% of the rounding interval has elapsed without visitation. An auditory alarm may be added as yet another threshold is passed. Such compliance indicia and compliance factors may be selected by the hospital staff based upon patient health status, departmental practice, physician orders, customary standard of care, or any combination thereof.

Using FIG. 1 again as an example, assuming room (102) had a rounding interval of one hour, the server, after determining the rounding status for room (102), would determine whether the nurse (110) having the wearable tag (120) had visited room (102) within the past hour. As such, if the rounding status depicted the nurse (110) having the wearable tag (120) entering room (102) within the past hour, the server shall be prompted to assign a color of green to room (102) depicted in the automated rounding report. If the rounding status depicted the nurse (110) last entering room (102) two hours ago, and the compliance factor for the “moderately non-compliant”) was set to twice the rounding interval, the server may assign a color of yellow to room (102) depicted in the automated rounding report. Still further, if the rounding status depicted the nurse last entering room (102) three hours ago and the compliance factor for the “urgent non-compliance” indicator is set to three times the rounding interval, the server shall assign a color of red to room (102) depicted in the automated rounding report. After another ten minutes without visitation, the feedback device might sound an auditory alarm. The server shall regularly perform all of the above processes for a patient shown in the HHS Census database until the patient is discharged and removed from the HHS Census database.

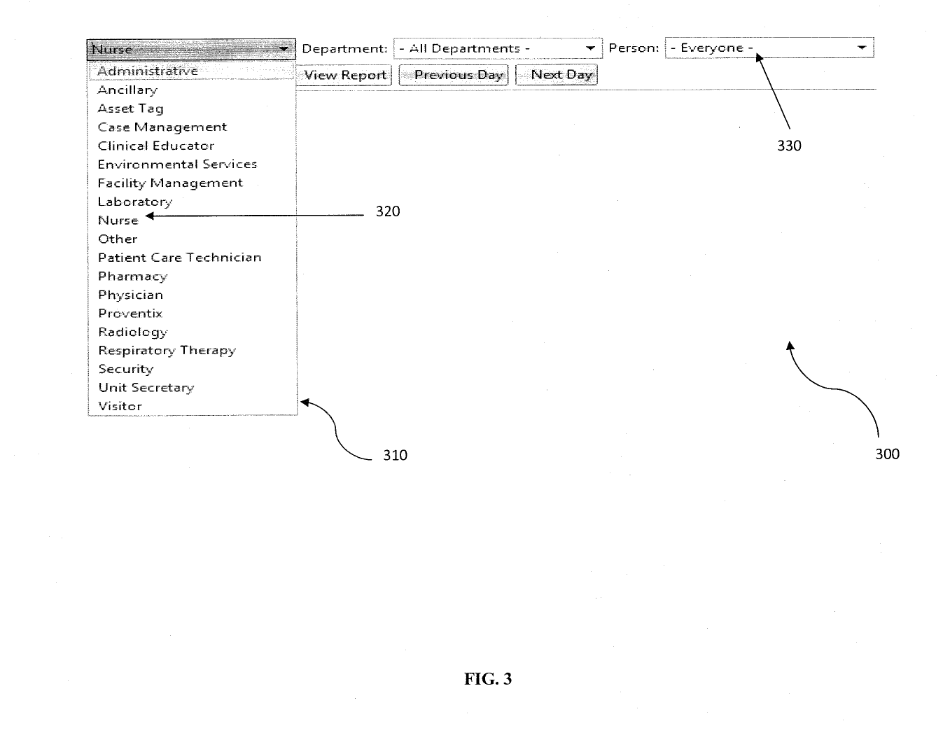

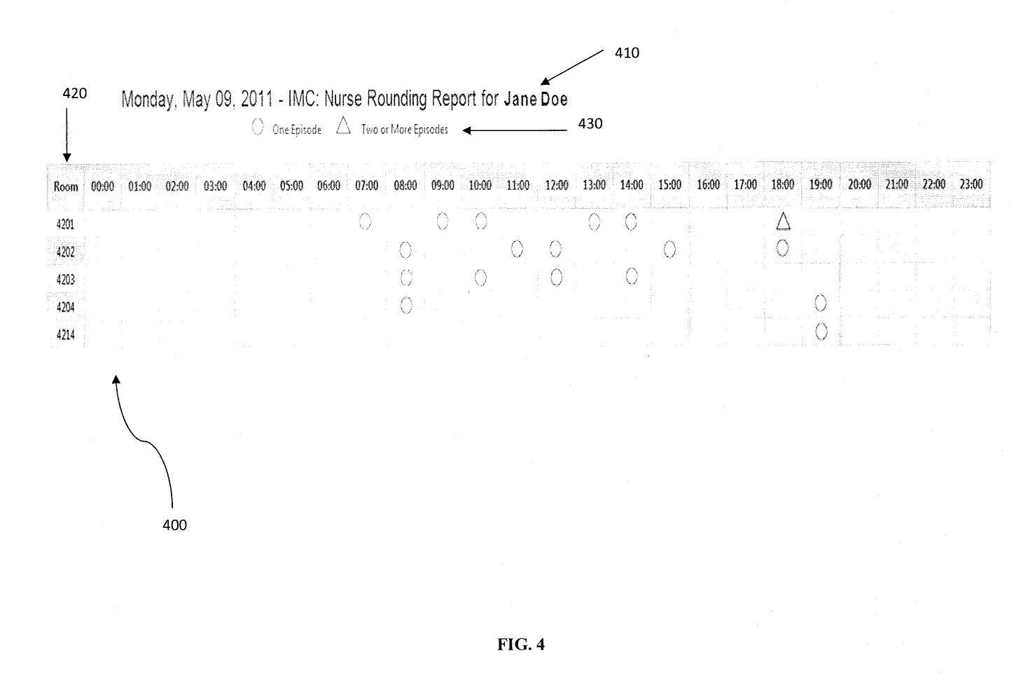

Turning to FIG. 3, a user requesting a report of compliance (300) with a hospital procedure can isolate the report of compliance (300) to a class of assets (310). Referring now to FIG. 1 and FIG. 3 in conjunction, the wearable tag (120) worn by the nurse (110) is operatively classified by the server within the class of assets (310) limited to a class of nurses (320). Therefore, once the user has isolated the report of compliance to the class of assets (310) which are of interest, in this instance, the class of assets (310) being limited to the class consisting of nurses (320), the feedback device (160) displays the report of compliance (i.e. rounding report) which is limited to the class of assets (310) pre-selected by the user. However, as demonstrated subsequently in FIG. 4, the user may further isolate the report of compliance (300) for the class of assets (310) to an individual asset (330) encompassed within the class of assets (310).

Referring now to FIG. 4 in conjunction with FIG. 3, a rounding report (400) is shown for a nurse asset (410) falling within the class of assets (310) limited to the class of nurses (320). The rounding report (400) depicts each of a plurality of rooms (420) where the nurse asset (410) has been in increments of an hour for an entire day. The rounding report (400) has the ability to illustrate a plurality of episodes (430), with the episodes (430) being instances where the nurse asset (410) was recognized entering a room on multiple occasions by a control unit during any given hour of the day.

FIG. 5 shows an exemplary automated rounding report (500) displayed on a feedback device (160) resulting from data gathered based upon the activities shown in FIG. 1 and the processes of FIG. 1. In one embodiment, the automated rounding report (500) depicts a grid displaying each of a plurality of rooms on a hospital floor equipped with the hand hygiene compliance system. As such, by performing the processes previously discussed in further detail in FIG. 2, the server assigns a compliance indicator to each of the rooms depicted in the automated rounding report (500). In a preferred embodiment, the compliance indicator is a color consisting of green, yellow, red, and white, as indicated by legend (510). In the automated rounding report (500), a room (520) currently unoccupied is assigned the color of white. A room (530) is assigned the color of green since the rounding status does not exceed the rounding interval assigned to the room. A room (540) is assigned the color of yellow since the rounding status exceeds the rounding interval by a factor of one. A room (550) is assigned the color of red since the rounding status exceeds the rounding interval by a factor of two. By assigning a color to each of the rooms depicted in the automated rounding report (500), the server generates a notification regarding compliance with patient rounding in response at least in part to data received from the wireless transmission (140) occurring between the control units (130) and the wearable tag (120).

Using FIG. 1 as an example to further illustrate the benefit of the automated rounding report (500) depicted in FIG. 5, the nurse (110) viewing the automated rounding report (500) on a feedback device (160) can readily identify a sequence in which rooms should be visited next to ensure compliance with the rounding interval established by a hospital. In FIG. 5, the room (550) assigned a color of red should be visited first by the nurse (110). Next, the nurse (110) must visit the room (540) assigned a color of yellow. The nurse (110) can go visit the room (530) assigned the color of green. However, the nurse (110) need not expend any time visiting the room (520) assigned a color of white since it is currently unoccupied. In this embodiment, the automated rounding report (500) not only reports existing compliance with hospital procedure, but also directs workflow and aids the nurse in predicting his/her next move using data collected by the hand hygiene compliance system to stay in compliance or remedy non-compliance an efficient manner.

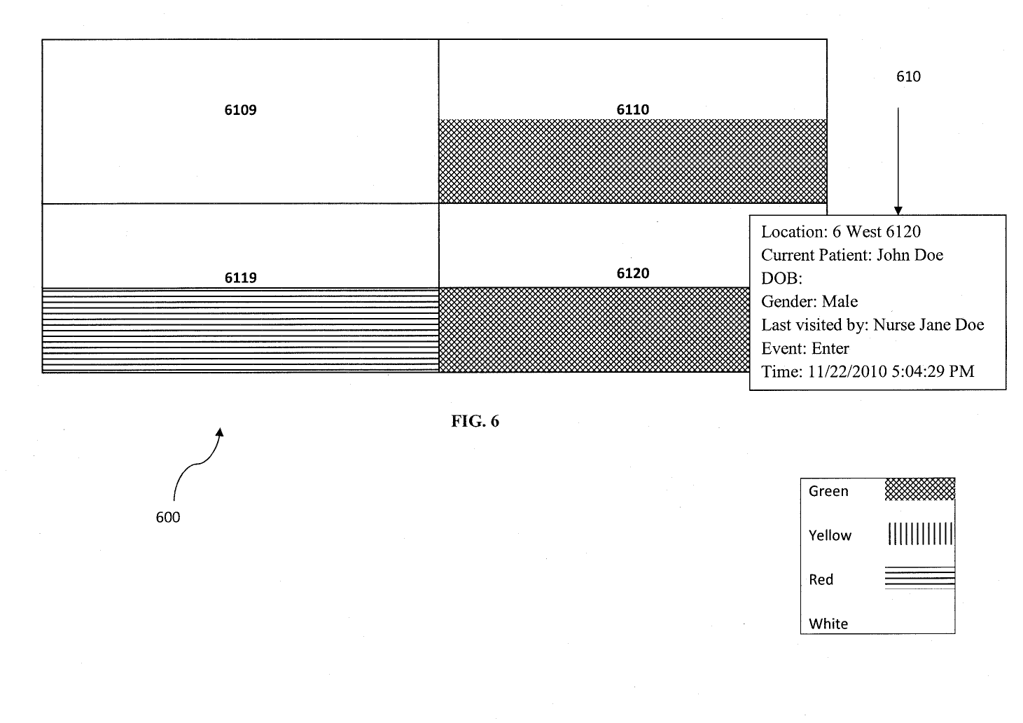

Turning now to FIG. 6 in conjunction with FIG. 1, an exploded view of an automated rounding report (600) is shown. By using data collected through the wireless transmission (140) occurring between the wearable tag (120) and the control units (130), a user viewing the automated rounding report (600) may ascertain a rounding status (610) associated with each of the rooms depicted. Using FIG. 1 as an illustrative example, the nurse (110) viewing the automated rounding report (600) on a feedback device (160) located at the nurses' station (150) may view the rounding status (610) associated with an individual patient. As previously stated, the rounding status (610) is comprised of data collected by the control units (130) during the wireless transmission (140) between the wearable tag (120) and the control units (130). The rounding status (610) allows a user viewing the automated rounding report (600) to ascertain the time of detection as well as the identity of the wearable tag (120) last seen in each of the rooms depicted in the automated rounding report (600).

In yet another embodiment of the present invention, a control unit may detect the presence a wearable tag associated with a piece of equipment (i.e. catheter), and in response to movement of the piece of equipment by a wearable tag associated with a person, the server may relay a procedure or a set of procedures relevant to the piece of equipment and the person on a feedback device associated with the control unit relating to proper administration of the piece of medical equipment and further requiring input by the person through the feedback device confirming compliance with the procedure or the set of procedures.

While an assortment of exemplary embodiments of the present invention have been disclosed for purposes of illustration, it is obvious that many modifications and variations could be made thereto. It is intended to cover all of those modifications and variations which fall within the scope of the present invention, as defined by the following claims.

1. A system comprising:
a hand hygiene compliance system comprising a plurality of control units, a plurality of wearable badges, and a server, each of said badges associated with an asset, each of said control units operable to detect and identify a badge within a predetermined proximity of said control unit and communicate data over a communications network to said server; and a feedback device;
wherein said server is operable to perform at least one of the following actions:
(a) generating a report of compliance with a hospital procedure based at least in part on said data in response to a query by a user and display the report on a feedback device;
(b) selecting at least one hospital procedure relevant to said data and providing workflow instructions to a person performing such procedure; or
(c) generating a notification regarding compliance with a procedure in response at least in part to said data.

2. The system of claim 1, wherein said asset is selected from the group consisting of:
(a) a person;
(b) a piece of equipment; and
(c) a supply.

3. The system of claim 2, wherein said data includes at least one of the following:
(a) the identity of the asset associated with the badge detected by a control unit;
(b) the location of said asset;
(c) the time of detection of said asset;
(d) movement of said asset; and
(e) signal strength of the communication between said badge and said control unit.

4. The system of claim 3, wherein the action performed is (c) and the procedure is a rounding interval for a patient.

5. The system of claim 4, wherein the notification comprises a rounding report displayed on a feedback device dynamically updated based upon data received from said control units.

6. The system of claim 5, wherein said system accesses hospital census data and said notification excludes from said rounding report any unoccupied room.

7. The system of claim 5, wherein said system accesses a patient information database and assigns a rounding interval specific to individual patients based upon records in said database.

8. The system of claim 5, wherein said feedback device is a display at a nurses' station.

9. The system of claim 5, wherein said notification directs workflow by identifying a sequence in which patient rooms should be visited to ensure compliance with said procedure.

10. A system comprising:
a hand hygiene compliance system comprising a plurality of control units, a plurality of wearable badges, and a server, each of said badges associated with a person, each of said control units operable to detect and identify said badges within a predetermined proximity of said control unit and communicate data over a communications network to said server; and a feedback device;
wherein said server is operable to perform at least the following actions:
(a) generating a rounding report based at least in part upon data received from said control units in order to monitor compliance with a rounding interval assigned to a patient resident at a healthcare facility; and
(b) displaying said rounding report on said feedback device, wherein said rounding report is dynamically updated in response at least in part to data received from said control units.

11. The system of claim 10, wherein said data includes at least one of the following:
(a) the identity of a person associated with one of said badges detected by one of said control units;
(b) the location of said person;
(c) the time of detection of said person;
(d) movement of said person; and
(e) signal strength of the communication between said badge and said control unit.

12. The system of claim 10, wherein said system accesses hospital census data and excludes from said report any unoccupied rooms.

13. The system of claim 10, wherein said system accesses a patient information database and assigns said rounding interval to said patient based upon records in said database.

14. The system of claim 10, wherein said feedback device is a display at a nurses' station.

15. The system of claim 10, wherein there are a plurality of patients, and said report directs workflow by identifying a sequence in which each of said plurality of patients should be visited to ensure compliance with a rounding interval specific to each of said plurality of patients.

16. A system comprising:
a hand hygiene compliance system comprising a plurality of control units and a plurality of badges, each of said control units operable to detect and identify a first badge associated with a piece of medical equipment and a second badge associated with a person within a predetermined proximity of said control unit and to display on a feedback device associated with said control unit workflow instructions for performing a procedure responsive to the identities of said person and said equipment.

17. The system of claim 16, wherein the system requires input from said person performing the procedure via said feedback device to confirm compliance with the procedure.

18. A method comprising:
identifying with a hand hygiene compliance (HHC) system one of a plurality of preselected persons within a predetermined proximity of a control unit of said HHC system, wherein said control unit corresponds to a patient;
recording the time when said person was within said proximity;
generating a rounding report to monitor compliance with a rounding interval associated with said patient based on said recorded time.

19. The method of claim 18, further comprising repeating said identifying and recording steps each time one of said persons comes within the predetermined proximity of said control unit and updating said rounding report based on the most recent recorded time.

20. The method of claim 19, further comprising:
displaying the rounding report on a feedback device wherein said report is updated at least in part each time one of said persons comes within the predetermined proximity of said control unit.

21. The method of claim 20, further comprising updating said report if none of said preselected persons comes within said proximity for a preselected time period.

22. The method of claim 21, wherein said preselected time period corresponds to a time period selected from the group consisting of: said rounding interval less a specified duration; said rounding interval; said rounding interval plus a specified duration.

23. The method of claim 22, wherein said feedback device provides a visual or auditory notification corresponding to each of said preselected time periods.

24. The method of claim 19, further comprising updating said report if none of said plurality of preselected persons comes within said proximity for a preselected time period.

25. The method of claim 19, wherein there are a plurality of patients and a plurality of control units, each said control unit corresponding to one of said plurality of patients, each said patient being associated with a rounding interval, and further comprising performing said identifying and recording steps with respect to each said control unit, and wherein said rounding report monitors compliance with the rounding interval associated with each said patient.

26. The method of claim 25, further comprising indicating on said rounding report a sequence in which at least one or more of said plurality of patients should be visited based upon said rounding interval associated with each said patient and the most recent recorded time when one of said plurality of persons was within said predetermined proximity of the control unit associated with said patient.

27. The method of claim 26, wherein said sequence is updated at least in part each time one of said control units identifies one of said preselected persons within a predetermined proximity of said control unit.
